(19) United States
(12) Patent Application Publication
(10) Pub. No.: US 2024/0343695 A1
(43) Pub. Date: Oct. 17, 2024

(54) Lysophosphatidic Acid Receptor 1 Antagonists as Migrastatics for Cancer

(71) Applicant: Lakehead University, Thunder Bay (CA)

(72) Inventors: Jinqiang Hou, Thunder Bay (CA); Wenjie Liu, Thunder Bay (CA)

(21) Appl. No.: 18/628,197

(22) Filed: Apr. 5, 2024

Related U.S. Application Data
(63) Continuation-in-part of application No. 18/588,717, filed on Feb. 27, 2024.
(60) Provisional application No. 63/488,599, filed on Mar. 6, 2023.

(51) Int. Cl. C07D 249/06; A61K 31/4192; A61K 31/4439; C07D 401/12

(52) U.S. Cl. CPC C07D 249/06; A61K 31/4192; A61K 31/4439; C07D 401/12

(57) ABSTRACT

Fluorine-containing triazole derivatives as potent LPA1 antagonists were successfully discovered. Among them, one compound demonstrated great inhibition activity of LPA-induced cell survival, migration, and invasion on a breast cancer cell line. Our findings further support the role LPA plays in breast cancer. These results provide a new alternative for the development of novel targeted therapy for the treatment of breast cancer.

### PRIOR APPLICATION INFORMATION

The instant application is a continuation-in-part of U.S. Ser. No. 18/588,717, filed Feb. 27, 2024, which claimed the benefit of U.S. Provisional Patent Application Ser. No. 63/488,599, filed Mar. 6, 2023, entitled “LYSOPHOSPHATIDIC ACID RECEPTOR ANTAGONISTS FOR BREAST CANCER TREATMENT”, now abandoned, the entire contents of which are hereby incorporated herein by reference for all purposes.

### BACKGROUND OF THE INVENTION

Cancer is a leading cause of death and a significant obstacle to increasing life expectancy worldwide. Cancer remains a leading cause of death worldwide, responsible for nearly 10 million deaths in 2020, representing nearly one in six deaths globally. Metastasis is responsible for about 90% of cancer deaths. Anti-metastatic drugs, termed as ‘migrastatics’, offer a distinctive therapeutic approach to address cancer migration and invasion. Migrastatics work by restricting the movement of cancer cells to surrounding tissues, unlike traditional cytotoxic drugs, which employ a direct cell-killing approach. While cytotoxic drugs inevitably induce drug resistance and promote the emergence of more aggressive cancer cells through the Darwinian selection of resistant clones that become the predominate cancer cell population, migrastatics will unlikely induce drug resistance because cancer cells are not directly stressed during treatment. Even if some cancer cells acquire resistance to migrastatics, they will not have the proliferative advantage nor give rise to destructive metastases. There is an urgent need to develop migrastatics to prevent metastasis in early-stage cancer and impede metastasis in advanced cancer. However, therapeutic exploitation of metastasis-specific targets remains limited, and the effective prevention and suppression of metastatic cancer continue to be elusive.

Breast cancer is the most commonly diagnosed cancer in the female population. Current breast cancer chemotherapies target multiple receptors such as estrogen receptor (ER), progesterone receptor (PR), and human epidermal growth factor receptor (HER2). However, around 12-17% of breast cancer patients are diagnosed with triple-negative breast cancer (TNBC), characterized by the absence of expression of the three aforementioned receptors. TNBC is the most aggressive histological subtype of breast cancer, with a poorer prognosis compared to other subtypes, due to the absence of targeted therapeutic options. Distant metastasis occurs in approximately 46% of TNBC patients. Therefore, the discovery of novel therapeutics targeting the TNBC metastasis is crucial for addressing this challenging issue.

Lysophosphatidic acid (LPA) is a bioactive phospholipid implicated in a diverse array of cellular activities that regulate cell survival, migration, invasion, and apoptosis (4). The most commonly used form of LPA in research is 18:1 LPA (1-oleoyl-2-hydroxy-sn-glycero-3-phosphate). Six LPA receptors (LPA₁₋₆) have been discovered so far, which belong to G protein-coupled receptors (GPCRs) families. Based on phylogeny, LPA receptor subtypes are divided into two categories: the endothelial gene (EDG) family, including LPA₁₋₃, and the non-EDG family, including LPA₄₋₆. LPA₁₋₃ share 45-56% sequence similarity, while LPA₄₋₆ share 35-55% sequence similarity (4). Dysregulation of LPA₁ is related to several types of cancer, including breast cancer (5, 6), ovarian cancer (7, 8), and cervical cancer (9). It has been reported that the expression of LPA₁ is elevated in the breast cancer cell line MDA-MB-231 (10), and silencing of LPA₁ expression significantly inhibited survival of breast cancer cell lines in vitro (6). Furthermore, animal studies showed that suppression of LPA₁ activity inhibited breast cancer bone metastases (6). This evidence shows that LPA₁ could be a promising migrastatic target in cancer, specifically in breast cancer with metastases. However, the number of LPA₁ ligands developed so far has been limited, and no subtype selective LPA₁ ligand has yet been discovered specifically for cancer therapy.

The initial ligands targeting LPA₁ were lipid-like molecules with a long fatty acid chain. These molecules usually displayed poor drug-like properties due to the hydrophobic moiety. The first non-lipid compound Ki16425 was reported to be an LPA₁/LPA₃ dual antagonist (11). Based on the isoxazole scaffold of Ki16425 (FIG. 1), other potent and selective LPA₁ antagonists were discovered, for example, BMS-986020 and ¹¹C-BMT-136088. BMS-986020 (FIG. 1) is currently under phase II clinical trials for the treatment of idiopathic pulmonary fibrosis (12). ¹¹C-BMT-136088 is the only LPA₁ radiotracer reported, and it is in a phase clinical trial (13, 14).

RO6842262 is another Ki16425-derived LPA₁ antagonist for the treatment of idiopathic pulmonary fibrosis. By substituting the isoxazole to the triazole scaffold, RO6842262 (FIG. 1) was found to be a potent and selective LPA₁ antagonist with an IC₅₀ value of 25 nM (LPA₃ IC₅₀>30 μM) in calcium release assay (15). RO684226 inhibited lung fibroblast proliferation and contraction induced by LPA in vitro, and ameliorated histamine release induced by LPA in vivo, demonstrating great therapeutic potential for lung fibrosis (15).

### SUMMARY OF THE INVENTION

According to a first aspect of the invention, there is provided a compound comprising a chemical structure according to Formula (I) or (II):

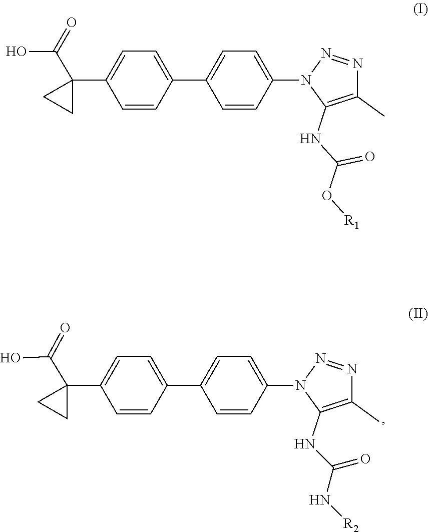

wherein R1 and R2 are independently a fluorocumene or a fluoroethylbenzene.

The compounds may be used as LPA₁ agonists.

The compounds may be used for treating an LPA₁-related pathological condition.

According to another aspect of the invention, there is provided a method of treating an LPA₁-related pathological condition comprising administering an effective amount of a compound as described above to an individual in need of such treatment.

### BRIEF DESCRIPTION OF THE DRAWINGS

FIG. 1. Structures of known LPA₁ antagonists. PRIOR ART.

FIG. 2. Effects of LPA and 1f on cell survival by colony formation assays in MDA-MB-231 cells. * P<0.05, ** P<0.01, **** P<0.0001, ns P>0.05.

FIG. 3. Effects of LPA and 1f on cell migration and invasion in MDA-MB-231 cells. A. Effects of LPA and 1f on cell migration by wound healing assays in MDA-MB-231 cells. B. Effects of LPA and 1f on cell migration and invasion by transwell assays in MDA-MB-231 cells. C-E. Quantification of wound healing (C), transwell migration (D), and transwell invasion (E) assays. Effects of 10 UM LPA and various concentrations of 1f on cell apoptosis in MDA-MB-231 cells (F). * P<0.05, ** P<0.01, *** P<0.001, **** P<0.0001, ns P>0.05.

FIG. 4. Synthesis of ethyl 1-(4-(4, 4, 5, 5-tetramethyl-1, 3, 2-dioxaborolan-2-yl) phenyl) cyclopropane-1-carboxylate.

FIG. 5. Synthesis of the 1^{st} series of compounds.

FIG. 6. Synthesis of the 2^{nd} series of compounds.

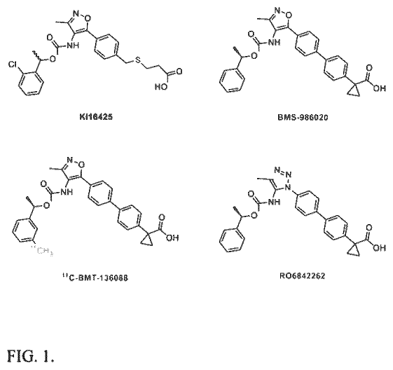

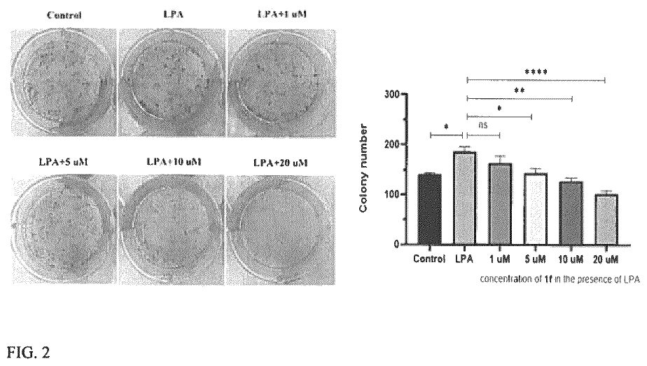

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

We used the structure of RO6842262 as a starting point, and incorporated fluorine into this compound to develop novel and potent LPA₁ antagonists for the treatment of cancer and potential use as PET imaging agents. We identified carbamates with a triazole scaffold as potent LPA₁ antagonists in CAMP assay. Particularly, as discussed below, compound 1f was the most potent in this series. Specifically, compound 1f inhibited LPA-induced survival, migration, and invasion in a breast cancer cell line MDA-MB-231 in a dose-dependent manner.

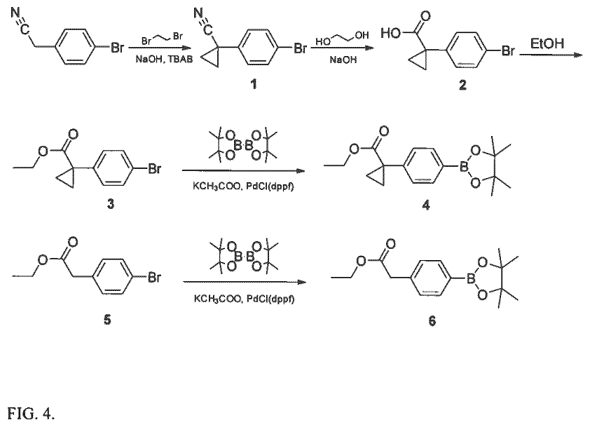

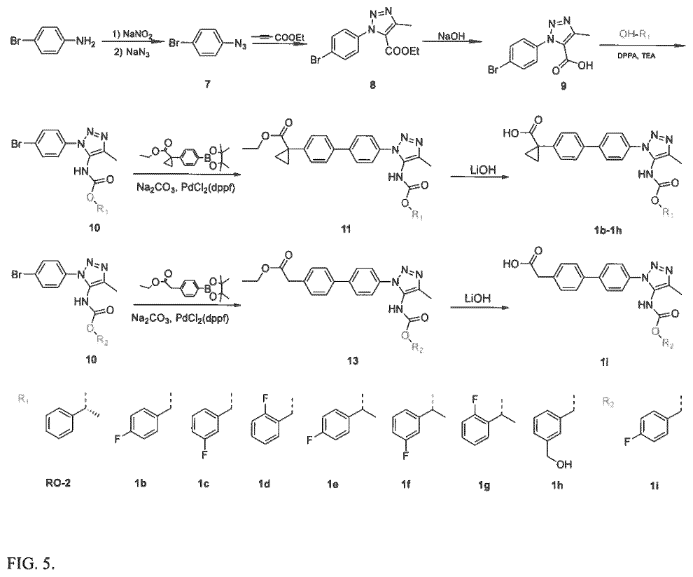

The 1^{st} Series of Triazole analogues were synthesized according to Scheme 1, shown in FIG. 4 and Scheme 2, shown in FIG. 5, which are similar to the reported methods.

Specifically, 4-Bromoaniline was reacted with sodium nitrite to afford 1-Azido-4-bromobenzene 1. Compound 1 was then treated with but-2-ynoic acid ethyl ester to form compound 2, which was further reacted with sodium hydroxide to form the corresponding carboxylic acid 3. Curtius rearrangement reaction was then performed with an aromatic hydroxy building block to form the intermediate compound 4, followed by the Suzuki-coupling reaction to afford compound 5. Last, the ester on compound 5 was hydrolysed to afford the final compounds 1b-1i, shown in FIG. 4.

As known to those of skill in the art, incorporation of fluorine into small molecules can enhance a number of pharmacokinetic and physicochemical properties such as improved metabolic stability and enhanced membrane permeation. Systematic fluorine substitution of ligands is a strategy frequently used in drug design and development.

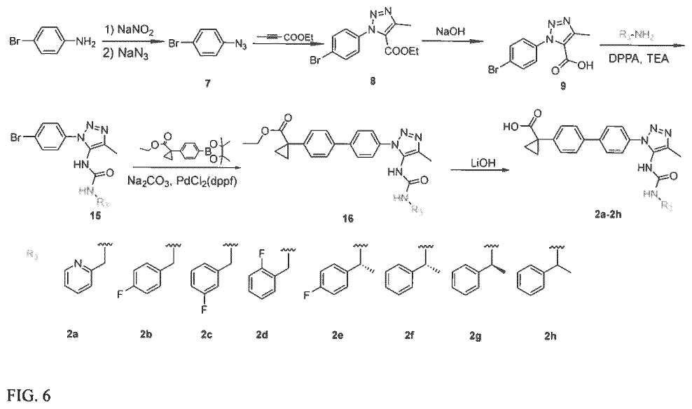

Based on the reaction mechanism of the Curtius rearrangement, we changed the aromatic hydroxy building block into the aromatic amine building block, and the 2^{nd} series of compound with a urea moiety were produced, as shown in FIGS. 5 and 6.

Specifically, we wanted to see if replacing O (hydrogen bond acceptor) with NH (hydrogen bond acceptor) would improve the functional activity, in part due to the fact that urea is able to form multiple stable hydrogen bonds with receptor targets. In our study, a urea moiety could be easily introduced by changing the aromatic hydroxy building block into the aromatic amine building block. Therefore, O was replaced with NH in an attempt to improve the ligand-receptor binding/interaction. In addition, urea is generally speaking more stable than carbamate.

However, the overall activity of the second series of compounds is lower compared with the first series of compounds, as discussed below.

LPA₁ receptor activation leads to an inhibition of cAMP production that can be quantified by measuring the luminescence. Compounds prepared as described herein were tested in an in vitro cAMP assay using a CHO cell line overexpressing LPA₁ receptor. The endogenous ligand 18:1 LPA was used at a final concentration of 1 μM to activate the LPA₁ receptor. The LPA₁ antagonist activity is presented as IC₅₀, which is the concentration to reverse 50% of the inhibitory effect of 18:1 LPA on forskolin-mediated CAMP production. These results are shown in Tables 1 and 2.

As can be seen in Tables 1 and 2, compound 1f showed an IC₅₀ value lower than the control compound.

Furthermore, as can be seen, the activities of 1c and 1f indicated that the removal of the methyl substitution at the alpha position of the carmate resulted in a dramatic decrease in the activity, which suggests that the methyl group plays an important role in the ligand-receptor interaction.

According to an aspect of the invention, there is provided a compound comprising or consisting of or consisting essentially of a chemical structure according to Formula (I) or (II):

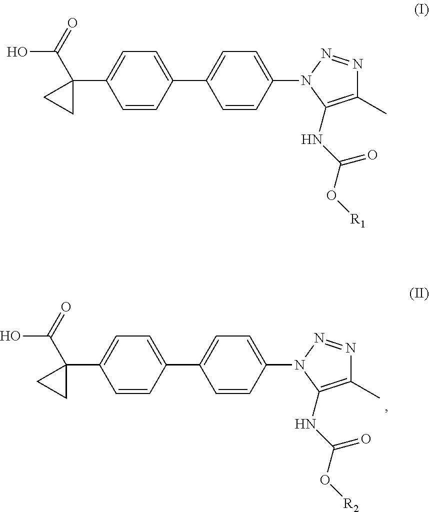

wherein R₁ and R₂ are independently a fluorocumene or a fluoroethylbenzene.

In some embodiments of the invention, R₁ and R₂ are independently selected from the group consisting of:

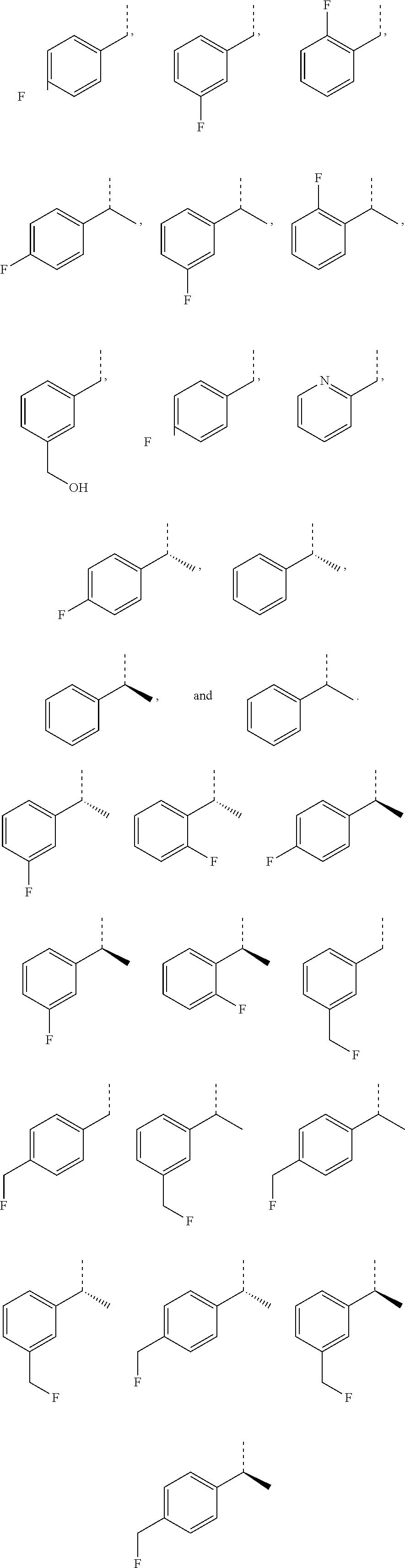

In some embodiments of the invention, R₁ and R₂ are independently selected from the group consisting of:

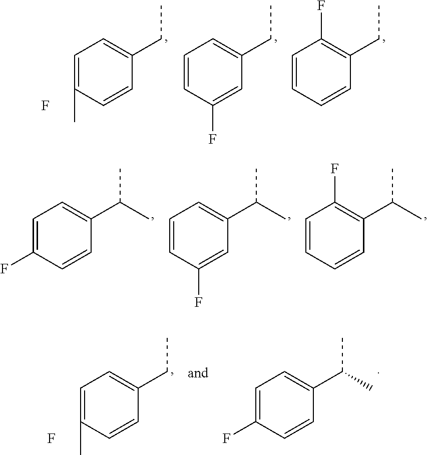

In some embodiments of the invention, the compound comprises or consists of or consists essentially of a chemical structure as set forth in Formula (I) and R₁ is selected from the group consisting of:

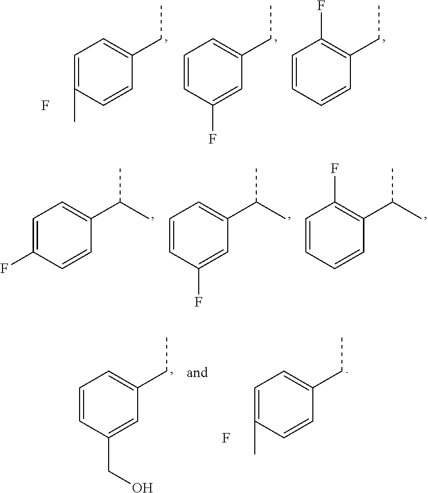

In some embodiments of the invention, the compound comprises or consists of or consists essentially of a chemical structure as set forth in Formula (II) and R₂ is selected from the group consisting of:

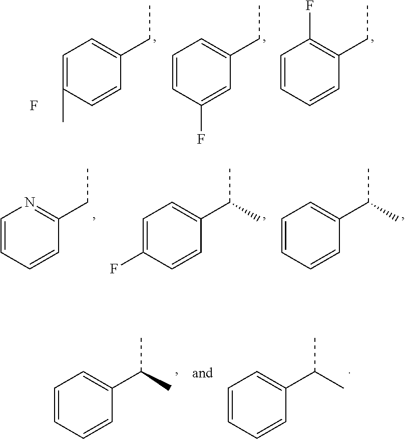

In some embodiments of the invention, the compound comprises or consists of or consists essentially of a chemical structure as set forth in Formula (I) and R₁ is

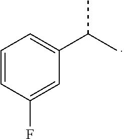

As will be appreciated by one of skill in the art, as used above, “consists essentially of” indicates that the compound may include additional substituents which have no statistically significant effect on the relevant activities, for example, the LPA₁ agonist activity, of the compound.

In some embodiments of the invention, the compounds as described above are used as LPA₁ agonists. Specifically, as discussed herein, the fluorinated compounds are LPA₁ antagonists as shown at least by cAMP assay. Accordingly, these LPA₁ antagonists are able to block LPA action on the LPA₁ receptor.

Furthermore, our goal was not only to create new treatment options for breast cancer but also to develop Fluorine-18 radio-labelled PET imaging agents for early diagnosis of breast cancer. Fluorine-18 is the most widely used isotope used in positron emission tomography (PET) and has a favorable half-life of 109.7 minutes. Thus, while RO684226 without a fluorine atom cannot be used as a ‘cold’ compound for Fluorine-18 tracer development, compound 1f can be radiolabelled with Fluorine-18 and used as a PET imaging tracer for early diagnosis of breast cancer.

Furthermore, the ligands can be used to treat other LPA₁-related pathological conditions, such as, for example, but by no means limited to, lung, glioblastoma, cervical cancer, and Idiopathic Pulmonary Fibrosis.

In order to study the effect of LPA and the compound 1f on the survival, migration, and invasion of MDA-MB-231, several in vitro assays were performed. Colony formation assay is a cell survival assay based on the ability of a single cell to grow into a colony and was performed to evaluate cell survival (17). Our results showed that LPA enhanced the survival of MDA-MB-231, while 1f inhibited LPA-induced MDA-MB-231 cell survival in a dose-dependent manner (FIG. 2).

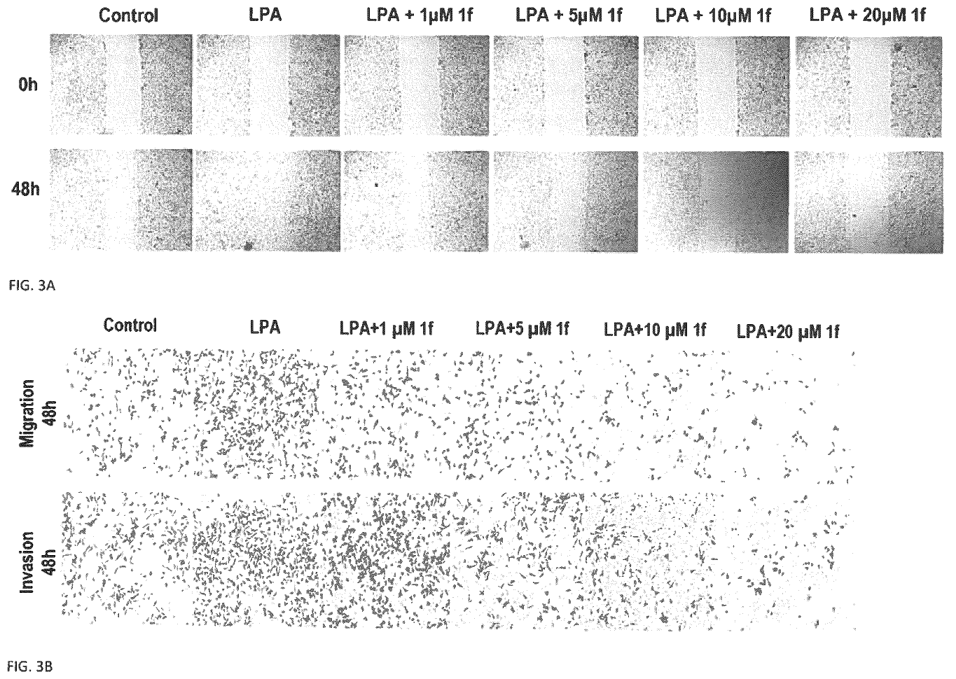

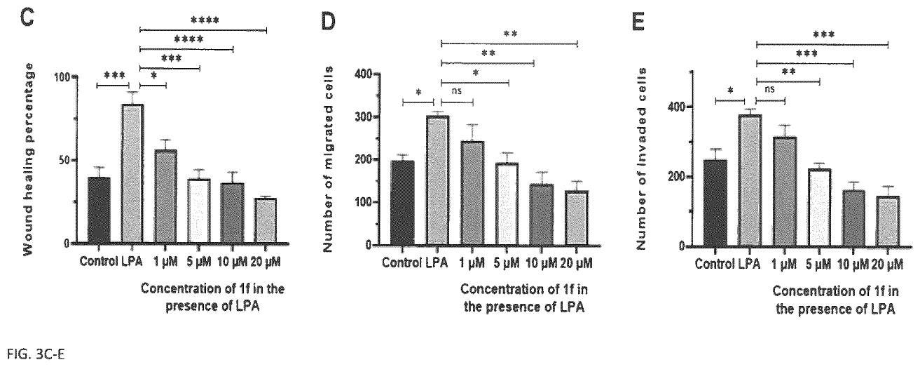

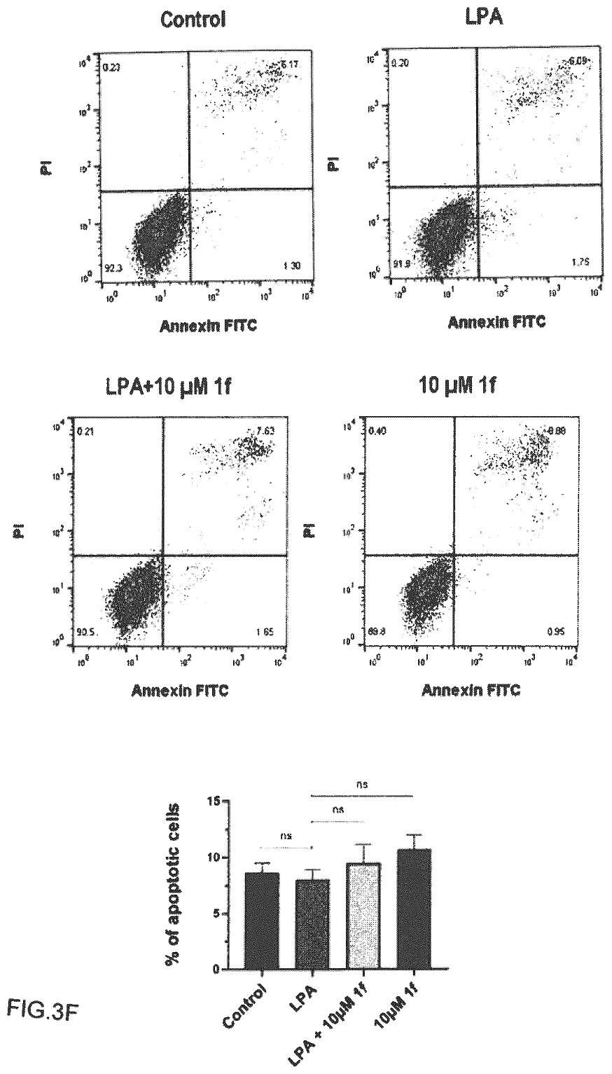

Wound healing assay is a well-established method to determine cell migration in vitro. Firstly, a scratch was created in the cell monolayer, and then cells migrate to close the scratch. Cell migration rate can be measured using this method. The results showed that stimulation with LPA enhanced cell migratory ability, while addition of 1f impeded cell migration dose-dependently (FIG. 3A).

In addition, transwell migration/invasion assays were performed to further confirm the effect of LPA and 1f on cell migration and invasion. Transwell assay is based on a chamber of two media-filled partitions separated by a microporous membrane. Cells were plated in the upper chamber and are allowed to migrate to the lower chamber through the membrane pores with the stimulation of LPA and 1f. For the invasion assay, a layer of basement membrane extract was precoated on the chamber to block the pores of the membrane and prevent non-invasive cells from migrating through the membranes. Invasive cells are able to secret proteases which degrade the membrane extract in order to invade through the membrane. The results from transwell assays suggested that LPA induced cell migration and invasion on MDA-MB-231, which was hindered by 1f dose-dependently (FIGS. 3B and 3C).

Cancer cells are recognized for their ability to evade apoptosis, enabling them to survive for longer periods. Next, we explored the impact of LPA and 1f on cell apoptosis. It was observed that LPA did not promote breast cancer cell apoptosis (FIG. 3F). The addition of 1f, either alone or together with LPA, displayed no effect on cell apoptosis.

In summary, fluorine-containing triazole derivatives as potent LPA₁ antagonists were successfully discovered. Among them, compound 1f demonstrated strong inhibition activity of LPA-induced cell survival, migration, and invasion on a breast cancer cell line, without inducing apoptosis. Our findings further support the role LPA/LPA₁ axis plays in breast cancer and LPA₁ antagonists are potential migrastatics that only inhibits the spread of tumor cells without killing them. These results provide a promising opportunity for the development of first-in-class therapy for the treatment of breast cancer metastasis.

Evaluation of lead compound 1f in lung cancer cell lines (A549) showed that 1f exhibited a dose-dependent inhibition of migration and invasion against lung cancer cells without observable anti-proliferative effect, suggesting that 1f could be used as a migrastatics for lung cancer. In addition, the novel migrastatics has the potential to be effective for other types of cancers (i.e., glioblastoma, cervical cancer) that may rely on the LPA/LPA₁ axis to metastasize.

The invention will now be further described by way of examples; however, the invention is not necessarily limited to or by the examples.

### EXAMPLE 1-Methods

### a) Synthesis of the 1^{st} Series of the Compounds

1-(4-Bromophenyl) cyclopropane-1-carbonitrile (1). Sodium hydroxide (3.05 g, 76.5 mmol) was dissolved in H₂O (5 mL) and toluene (12 mL). 4-bromophenylacetonitrile (1.5 g, 7.65 mmol) and tetrabutylammonium bromide (0.12 3 g, 0.38 mmol) was added, followed by dibromoethane (0.975 mL, 11.4 mmol) dropwise. The reaction was stirred at 85° C. for 5 hours. The reaction mixture was partitioned between DCM and water. Organic layer was extracted with DCM and washed with 1N HCl and brine, dried over Na₂SO₄, filtered and concentrated. The residue was purified by column chromatography (Hexane: EtOAc=20:1) to give compound 1 (523.33 mg, yield: 30.85%). ¹H NMR (500 MHZ, Chloroform-d) δ 7.50-7.45 (m, 2H), 7.19-7.13 (m, 2H), 1.77-1.71 (m, 2H), 1.41-1.34 (m, 2H).

1-(4-Bromophenyl) cyclopropane-1-carboxylic acid (2). Compound 1 (1.5 g, 6.75 mmol) and sodium hydroxide (1.07 g, 26.80 mmol) were dissolved in ethylene glycol (21 ml), and the reaction was stirred at 180° C. for 5 hours. The mixture was added with water and acidified with concentrated HCl, filtered under reduced pressure to give compound 2 (1.53 g, yield: 98%).

1-(4-Bromophenyl) cyclopropane-1-carboxylic acid ethyl ester (3). Compound 2 (1.53 g, 5.20 mmol) was dissolved in anhydrous EtOH (20 ml) and concentrated H₂SO₄ (1 ml) was added. The reaction was stirred at 80° C. for 9 hours. The mixture was diluted with EtOAc, washed with brine, dried over Na₂SO₄, filtered and concentrated to give compound 3 (1.10 g, yield: 80.88%). ¹H NMR (500 MHZ, Chloroform-d) δ 7.46-7.39 (m, 2H), 7.25-7.17 (m, 2H), 4.09 (qd, J=7.1, 0.4 Hz, 2H), 1.60 (d, J=3.0 Hz, 2H), 1.20-1.09 (m, 5H).

Ethyl 1-(4-(4, 4, 5, 5-tetramethyl-1, 3, 2-dioxaborolan-2-yl) phenyl) cyclopropane-1-carboxylate (4). The mixture of compound 3 (200 mg, 0.75 mmol), bis(pinacolato)diboron (208.45 mg, 0.825 mmol), potassium acetate (184.03 mg, 1.875 mmol) was dissolved in dry dioxane (2 ml), and PdCl₂ (dppf) (54.88 mg, 0.075 mmol) was added. The mixture was bubbled with argon and then stirred at 100° C. under argon for 6 hours. The mixture was filtered through diatomaceous earth and partitioned between EtOAc and water. The organic phase was extracted with EtOAc, washed with brine, dried over Na₂SO₄, filtered and concentrated. The residue was purified by column chromatography (Hexane: EtOAc=20:1) to give compound 4 (149 mg, yield: 63.15%). ¹H NMR (500 MHZ, Chloroform-d) δ 7.76 (d, J=7.6 Hz, 2H), 7.34 (d, J=7.6 Hz, 2H), 4.07 (q, J=7.1 Hz, 2H), 1.59 (q, J=4.0 Hz, 2H), 1.33 (s, J=1.7 Hz, 12H), 1.20-1.09 (m, 5H).

Ethyl 2-(4-(4, 4, 5, 5-tetramethyl-1, 3, 2-dioxaborolan-2-yl) phenyl) acetate (6). The mixture of compound 5 (1 g, 4.11 mmol), bis(pinacolato)diboron (1.149 mg, 4.52 mmol), potassium acetate (1.008 g, 10.28 mmol) was dissolved in dry dioxane (10 ml), and PdCl₂ (dppf) (0.301 g, 0.411 mmol) was added. The mixture was bubbled with argon and then stirred at 100° C. under argon for 6 hours. The mixture was filtered through diatomaceous earth and partitioned between EtOAc and water. The organic phase was extracted with EtOAc, washed with brine, dried over Na₂SO₄, filtered and concentrated. The residue was purified by column chromatography (Hexane: EtOAc=20:1) to give compound 6 (828 mg, yield: 69%). ¹H NMR (500 MHZ, Chloroform-d) δ 7.80-7.74 (m, 2H), 7.32-7.26 (m, 2H), 4.13 (q, J=7.1 Hz, 2H), 3.62 (s, 2H), 1.33 (s, 12H), 1.23 (t, J=7.1 Hz, 3H).

1-Azido-4-bromobenzene (7). 4-Bromoaniline (2 g, 11.63 mmol) was dissolved in 4N HCl (15 ml) and was cooled to 0° C. and stirred. To this suspension was added sodium nitrite (0.883 g, 12.79 mmol) dissolved in water (2.33 ml) dropwise. After 30 minutes, sodium azide (0.907 g, 13.95 mmol) was added portion-wise and the reaction mixture was slowly warmed to room temperature. And stirred for 1 hour. The reaction mixture was stored at 4° C. overnight and the resulting residue was filtered to give compound 7 (2.06 g, yield: 89.57%). ¹H NMR (500 MHZ, Chloroform-d) δ 7.52-7.39 (m, 2H), 6.98-6.82 (m, 2H).

Ethyl 1-(4-bromophenyl)-4-methyl-1H-1, 2, 3-triazole-5-carboxylate (8). To a solution of compound 7 (200 mg, 1.01 mmol) in toluene (1 ml) was added but-2-ynoic acid ethyl ester (0.14 ml, 1.21 mmol). The reaction mixture was heated to reflux at 110° C. under argon for 15 hours. The reaction mixture was concentrated and purified by column chromatography (Hexane: EtOAc=10:1) to give compound 8 (85 mg, yield: 27.16%). ¹H NMR (500 MHZ, Chloroform-d) δ 7.67-7.62 (m, 2H), 7.34-7.30 (m, 2H), 4.29 (q, J=7.1 Hz, 2H), 2.63 (s, 3H), 1.27 (t, J=7.1 Hz, 3H).

Ethyl 1-(4-bromophenyl)-4-methyl-1H-1, 2, 3-triazole-5-carboxylic acid (9). To a solution of compound 8 (80 mg, 0.26 mmol) in MeOH/THF/H₂O (0.6/0.6/0.6 ml) was added sodium hydroxide (51.72 mg, 1.29 mmol). The reaction mixture was stirred at room temperature overnight. The mixture was cooled down to 0° C. and neutralized to pH=4.0 with 3N HCl. The mixture was extracted with EtOAc and the organic phase was dried over Na₂SO₄, filtered and concentrated to give crude compound 9 (70 mg, yield: 96.21%).

2-Phenylpropan-2-yl (1-(4-bromophenyl)-4-methyl-1H-1, 2, 3-triazol-5-yl) carbamate (10). To a solution of compound 9 (70 mg, 0.248 mmol) in anhydrous toluene (1.75 ml) was added (4-fluorophenyl) methanol (40.36 mg, 0.32 mmol), triethylamine (50 mg), and DPPA (89.25 mg, 0.32 mmol). The reaction mixture was heated to 120° C. for 5 hours. The mixture was diluted with EtOAc, washed with brine, dried over Na₂SO₄, filtered and concentrated. The residue was purified by column chromatography (Hexane: EtOAc=5:1) to give compound 10 (50 mg, yield: 49.8%). ¹H NMR (500 MHZ, Chloroform-d) δ 7.58 (d, J=8.1 Hz, 2H), 7.33 (d, J=8.3 Hz, 4H), 7.05 (s, 2H), 5.09 (s, 2H), 2.31 (s, 3H).

Ethyl 1-(4′-(5-((((4-fluorobenzyl)oxy)carbonyl)amino)-4-methyl-1H-1,2,3-triazol-1-yl)-[1,1′-biphenyl]-4-yl) cyclopropane-1-carboxylate (11). To a stirred mixture of compound 10 (100 mg, 0.25 mmol), compound 4 (93.6 mg, 0.30 mmol), and sodium carbonate (55.92 mg, 0.53 mmol) in dioxane (6 ml) and water (2 ml) was added and PdCl₂ (dppf) (18.27 mg, 0.025 mmol). The mixture was bubbled with argon and then stirred at 80° C. under argon for 6 hours. The mixture was filtered through diatomaceous earth and partitioned between EtOAc and water. The organic phase was extracted with EtOAc, washed with brine, dried over Na₂SO₄, filtered and concentrated. The residue was purified by column chromatography (DCM:MeOH=50:1) to give compound 11 (102 mg, yield: 65.88%). ¹H NMR (500 MHZ, Chloroform-d) δ 7.63 (d, J=8.1 Hz, 2H), 7.57-7.38 (m, 6H), 7.26 (s, 2H), 6.99 (s, 2H), 6.67 (s, 1H), 5.09 (s, 2H), 4.12 (q, J=7.1 Hz, 2H), 2.29 (s, 3H), 1.65 (q, J=4.0 Hz, 2H), 1.23 (q, J=4.0 Hz, 2H), 1.19 (t, J=7.1 Hz, 3H).

1-(4′-(5-((((4-fluorobenzyl)oxy)carbonyl)amino)-4-methyl-1H-1,2,3-triazol-1-yl)-[1,1′-biphenyl]-4-yl) cyclopropane-1-carboxylic acid (1b). To a stirred solution of compound 11 (100 mg, 0.19 mmol) in THF (9.9 ml) and water (3.3 ml) was added lithium hydroxide (23.95 mg, 0.97 mmol). The reaction mixture was stirred at room temperature for 24 hours. The reaction mixture was cooled down to 0° C. and neutralized with concentrated HCl to pH=2. The mixture was extracted with EtoAc. The combined organic phase was dried over Na₂SO₄, filtered and concentrated. The residue was purified by column chromatography (Hexane: EtOAc=1:1) to give compound 1b (80 mg, yield: 84.61%). ¹H NMR (500 MHZ, Methanol-d4) δ 7.76 (d, J=8.3 Hz, 2H), 7.68-7.59 (m, 2H), 7.54 (d, J=8.1 Hz, 2H), 7.51-7.43 (m, 2H), 7.34 (s, 2H), 7.02 (s, 2H), 5.10 (s, 2H), 2.26 (s, 3H), 1.60 (q, J=3.9 Hz, 2H), 1.21 (q, J=4.0 Hz, 2H). ESI-MS m/z calculated C₂₇H₂₃FN₄O₄ [M+H]⁺ 487.18, found 487.26.

1-(4′-(5-((((3-fluorobenzyl)oxy)carbonyl)amino)-4-methyl-1H-1,2,3-triazol-1-yl)-[1,1′-biphenyl]-4-yl) cyclopropane-1-carboxylic acid (1c). Compound 1c was prepared in a similar way as compound 1b, except that (3-fluorophenyl) methanol was used from step 4 in scheme 2. ¹H NMR (500 MHZ, Methanol-d4) δ 7.77 (d, J=7.8 Hz, 2H), 7.63 (d, J=7.7 Hz, 2H), 7.56 (d, J=7.7 Hz, 2H), 7.49 (d, J=7.7 Hz, 2H), 7.30 (s, 1H), 7.04 (m, 3H), 5.14 (s, 2H), 2.28 (s, 3H), 1.62 (q, J=3.9 Hz, 2H), 1.25 (q, J=4.0 Hz, 2H). ESI-MS m/z calculated C₂₇H₂₃FN₄O₄ [M+H]⁺ 487.18, found 486.97.

1-(4′-(5-((((2-fluorobenzyl)oxy)carbonyl)amino)-4-methyl-1H-1,2,3-triazol-1-yl)-[1,1′-biphenyl]-4-yl) cyclopropane-1-carboxylic acid (1d). Compound 1d was prepared in a similar way as compound 1b, except that (2-fluorophenyl) methanol was used from step 4 in scheme 2. ¹H NMR (500 MHZ, Methanol-d4) δ 7.76 (d, J=7.9 Hz, 2H), 7.63 (d, J=7.8 Hz, 2H), 7.55 (d, J=6.8 Hz, 2H), 7.49 (d, J=7.7 Hz, 2H), 7.31 (d, J=6.0 Hz, 2H), 7.08 (s, 2H), 5.20 (s, 2H), 2.27 (s, 3H), 1.63 (q, J=3.9 Hz, 2H), 1.25 (q, J=4.0 Hz, 2H). ESI-MS m/z calculated C₂₇H₂₃FN₄O₄ [M+H]⁺ 487.18, found 487.28.

1-(4′-(5-(((1-(4-fluorophenyl)ethoxy)carbonyl)amino)-4-methyl-1H-1,2,3-triazol-1-yl)-[1,1′-biphenyl]-4-yl) cyclopropane-1-carboxylic acid (1e). Compound 1e was prepared in a similar way as compound 1b, except that 1-(4-fluorophenyl) ethan-1-ol was used from step 4 in scheme 2. ¹H NMR (500 MHZ, Methanol-d4) δ 7.74 (d, J=6.8 Hz, 2H), 7.61 (d, J=7.7 Hz, 2H), 7.58-7.42 (m, 4H), 7.34 (s, 2H), 7.02 (s, 2H), 5.72 (s, 1H), 2.24 (s, 3H), 1.62 (q, J=3.9 Hz, 2H), 1.50 (s, 3H), 1.25 (q, J=4.0 Hz, 2H). ESI-MS m/z calculated C₂₈H₂₅FN₄O₄ [M+H]⁺ 501.19, found 501.13.

1-(4′-(5-(((1-(3-fluorophenyl)ethoxy)carbonyl)amino)-4-methyl-1H-1,2,3-triazol-1-yl)-[1,1′-biphenyl]-4-yl) cyclopropane-1-carboxylic acid (1f). Compound 1f was prepared in a similar way as compound 1b, except that 1-(3-fluorophenyl) ethan-1-ol was used from step 4 in scheme 2. ¹H NMR (500 MHZ, Methanol-d4) δ 7.64 (d, J=8.1 Hz, 2H), 7.57-7.49 (m, 2H), 7.48-7.30 (m, 4H), 7.19 (s, 1H), 6.85 (td, J=8.6, 2.6 Hz, 3H), 5.62 (s, 1H), 2.15 (s, 3H), 1.52 (q, J=3.9 Hz, 2H), 1.41 (s, 3H), 1.15 (q, J=4.0 Hz, 2H). ESI-MS m/z calculated C₂₈H₂₅FN₄O₄ [M+H]⁺ 501.19, found 501.27.

1-(4′-(5-(((1-(2-fluorophenyl)ethoxy)carbonyl)amino)-4-methyl-1H-1,2,3-triazol-1-yl)-[1,1′-biphenyl]-4-yl) cyclopropane-1-carboxylic acid (1g). Compound 1g was prepared in a similar way as compound 1b, except that 1-(2-fluorophenyl) ethan-1-ol was used from step 4 in scheme 2. ¹H NMR (500 MHZ, Methanol-d4) δ 7.64 (d, J=8.1 Hz, 2H), 7.58-7.49 (m, 2H), 7.49-7.33 (m, 4H), 7.27 (s, 1H), 7.20-7.08 (m, 1H), 6.94 (m, 2H), 5.88 (s, 1H), 2.15 (s, 3H), 1.52 (q, J=3.9 Hz, 2H), 1.43 (s, 3H), 1.15 (q, J=4.0 Hz, 2H). ESI-MS m/z calculated C₂₈H₂₅FN₄O₄ [M+H]⁺ 501.19, found 500.83.

1-(4′-(5-((((3-ethylbenzyl)oxy)carbonyl)amino)-4-methyl-1H-1,2,3-triazol-1-yl)-[1,1′-biphenyl]-4-yl) cyclopropane-1-carboxylic acid (1 h). Compound 1h was prepared in a similar way as compound 1b, except that (3-ethylphenyl) methanol was used from step 4 in scheme 2. ¹H NMR (500 MHZ, Methanol-d4) δ 7.63 (d, J=8.2 Hz, 2H), 7.50 (d, J=8.0 Hz, 2H), 7.43 (d, J=8.0 Hz, 2H), 7.37 (d, J=8.0 Hz, 2H), 7.16 (d, J=4.2 Hz, 4H), 5.02 (s, 2H), 4.44 (s, 2H), 2.16 (s, 3H), 1.50 (q, J=4.0 Hz, 2H), 1.12 (q, J=4.0 Hz, 2H). ESI-MS m/z calculated C₂₉H₂₈N₄O₄ [M+H]⁺ 499.20, found 499.28.

2-(4′-(5-((((4-fluorobenzyl)oxy)carbonyl)amino)-4-methyl-1H-1,2,3-triazol-1-yl)-[1,1′-biphenyl]-4-yl) acetic acid (1i). Compound 1i was prepared in a similar way as compound 1b, except that compound 6 was used from step 5 in scheme 2. ¹H NMR (500 MHZ, Methanol-d4) δ 7.77 (d, J=8.1 Hz, 2H), 7.71-7.61 (m, 2H), 7.55 (d, J=8.1 Hz, 2H), 7.43 (d, J=7.7 Hz, 2H), 7.34 (s, 2H), 7.03 (s, 2H), 5.11 (s, 2H), 3.68 (s, 2H), 2.27 (s, 3H). ESI-MS m/z calculated C₂₅H₂₁FN₄O₄ [M+H]⁺ 461.16, found 461.27.

### b) Synthesis of the 2^{nd} Series of the Compounds

1-(4′-(4-methyl-5-(3-(pyridin-2-ylmethyl) ureido)-1H-1,2,3-triazol-1-yl)-[1,1′-biphenyl]-4-yl) cyclopropane-1-carboxylic acid (2a). Compound 2a was prepared in a similar way as compound 1b, except that pyridin-2-ylmethanamine was used from step 4 in scheme 2. ¹H NMR (500 MHZ, Methanol-d4) δ 8.49 (s, 1H), 7.77 (dd, J=18.4, 8.2 Hz, 3H), 7.64 (t, J=7.0 Hz, 4H), 7.51 (d, J=7.6 Hz, 2H), 7.30 (t, J=9.9 Hz, 2H), 4.64 (s, 2H), 2.53 (s, 3H), 1.64 (q, J=3.9 Hz, 2H), 1.27 (q, J=4.0 Hz, 2H). ESI-MS m/z calculated C₂₆H₂₄N₆O₃ [M+H]⁺ 469.20, found 468.63.

1-(4′-(5-(3-(4-fluorobenzyl) ureido)-4-methyl-1H-1,2,3-triazol-1-yl)-[1,1′-biphenyl]-4-yl) cyclopropane-1-carboxylic acid (2b). Compound 2b was prepared in a similar way as compound 1b, except that (4-fluorophenyl) methanamine was used from step 4 in scheme 2. ¹H NMR (500 MHZ, Methanol-d4) δ 7.82-7.72 (m, 2H), 7.67-7.55 (m, 4H), 7.53-7.44 (m, 2H), 7.17 (dd, J=8.5, 5.6 Hz, 2H), 6.94 (t, J=8.8 Hz, 2H), 4.26 (s, 2H), 2.28 (s, 3H), 1.60 (q, J=3.9 Hz, 2H), 1.23 (q, J=4.0 Hz, 2H). ESI-MS m/z calculated C₂₇H₂₄FN₅O₃ [M+H]⁺ 486.19, found 486.45.

1-(4′-(5-(3-(3-fluorobenzyl) ureido)-4-methyl-1H-1,2,3-triazol-1-yl)-[1,1′-biphenyl]-4-yl)cyclopropane-1-carboxylic acid (2c). Compound 2c was prepared in a similar way as compound 1b, except that (3-fluorophenyl) methanamine was used from step 4 in scheme 2. ¹H NMR (500 MHZ, Methanol-d4) δ 7.77 (d, J=8.1 Hz, 2H), 7.61 (dd, J=8.1, 5.3 Hz, 4H), 7.48 (d, J=7.9 Hz, 2H), 7.22 (dd, J=8.0, 5.9 Hz, 1H), 7.03-6.84 (m, 3H), 4.30 (s, 2H), 2.28 (s, 3H), 1.61 (q, J=4.0 Hz, 2H), 1.24 (q, J=2.9, 2.2 Hz, 2H). ESI-MS m/z calcd C₂₇H₂₄FN₅O₃ [M+H]⁺ 486.19, found 486.38.

1-(4′-(5-(3-(2-fluorobenzyl) ureido)-4-methyl-1H-1,2,3-triazol-1-yl)-[1,1′-biphenyl]-4-yl) cyclopropane-1-carboxylic acid (2d). Compound 2d was prepared in a similar way as compound 1b, except that (2-fluorophenyl) methanamine was used from step 4 in scheme 2. ¹H NMR (500 MHZ, Methanol-d4) δ 7.82-7.71 (m, 2H), 7.66-7.56 (m, 4H), 7.53-7.44 (m, 2H), 7.26-7.10 (m, 2H), 7.07-6.96 (m, 2H), 4.35 (s, 2H), 2.28 (s, 3H), 1.61 (q, J=3.9 Hz, 2H), 1.24 (q, J=4.0 Hz, 2H). ESI-MS m/z calculated C₂₇H₂₄FN₅O₃ [M+H]⁺ 486.19, found 486.23.

(R)-1-(4′-(5-(3-(1-(4-fluorophenyl)ethyl) ureido)-4-methyl-1H-1,2,3-triazol-1-yl)-[1,1′-biphenyl]-4-yl) cyclopropane-1-carboxylic acid (2e). Compound 2e was prepared in a similar way as compound 1b, except that (R)-1-(4-fluorophenyl) ethan-1-amine was used from step 4 in scheme 2. ¹H NMR (500 MHZ, Methanol-d4) δ 7.62 (d, J=8.2 Hz, 2H), 7.49 (d, J=8.0 Hz, 2H), 7.44 (d, J=8.2 Hz, 2H), 7.37 (d, J=7.9 Hz, 2H), 7.14 (dd, J=8.5, 5.3 Hz, 2H), 6.86 (t, J=8.6 Hz, 2H), 4.70 (q, J=7.0 Hz, 1H), 2.14 (s, 3H), 1.50 (q, J=3.9 Hz, 2H), 1.28 (d, J=7.1 Hz, 3H), 1.12 (t, J=3.5 Hz, 2H). ESI-MS m/z calculated C₂₈H₂₆FN₅O₃ [M+H]⁺ 500.21, found 500.39.

(R)-1-(4′-(4-methyl-5-(3-(1-phenylethyl) ureido)-1H-1,2,3-triazol-1-yl)-[1,1′-biphenyl]-4-yl) cyclopropane-1-carboxylic acid (2f). Compound 2f was prepared in a similar way as compound 1b, except that (R)-1-phenylethan-1-amine was used from step 4 in scheme 2. ¹H NMR (500 MHZ, Methanol-d4) δ 7.78-7.67 (m, 2H), 7.62-7.58 (m, 2H), 7.58-7.52 (m, 2H), 7.52-7.44 (m, 2H), 7.30-7.22 (m, 4H), 7.22-7.15 (m, 1H), 4.82 (q, J=7.0 Hz, 1H), 2.25 (s, 3H), 1.59 (q, J=3.8 Hz, 2H), 1.41 (d, J=7.0 Hz, 3H), 1.19 (q, J=3.9 Hz, 2H). ESI-MS m/z calculated C₂₈H₂₇N₅O₃ [M+H]⁺ 482.22, found 481.98.

(S)-1-(4′-(4-methyl-5-(3-(1-phenylethyl) ureido)-1H-1,2,3-triazol-1-yl)-[1,1′-biphenyl]-4-yl) cyclopropane-1-carboxylic acid (2g). Compound 2g was prepared in a similar way as compound 1b, except that(S)-1-phenylethan-1-amine was used from step 4 in scheme 2. ¹H NMR (500 MHZ, Methanol-d4) δ 7.78-7.68 (m, 2H), 7.64-7.58 (m, 2H), 7.58-7.52 (m, 2H), 7.52-7.44 (m, 2H), 7.31-7.22 (m, 4H), 7.22-7.15 (m, 1H), 4.83 (q, J=7.0 Hz, 1H), 2.25 (s, 3H), 1.63 (q, J=3.9 Hz, 2H), 1.41 (d, J=7.0 Hz, 3H), 1.25 (q, J=4.0 Hz, 2H). ESI-MS m/z calculated C₂₈H₂₇N₅O₃ [M+H]⁺ 482.22, found 482.42.

1-(4′-(4-methyl-5-(3-(1-phenylethyl) ureido)-1H-1,2,3-triazol-1-yl)-[1,1′-biphenyl]-4-yl) cyclopropane-1-carboxylic acid (2h). Compound 2h was prepared in a similar way as compound 1b, except that 1-phenylethan-1-amine was used from step 4 in scheme 2. ¹H NMR (500 MHZ, Methanol-d4) δ 7.78-7.69 (m, 2H), 7.63-7.58 (m, 2H), 7.58-7.53 (m, 2H), 7.51-7.45 (m, 2H), 7.30-7.22 (m, 4H), 7.22-7.15 (m, 1H), 4.83 (q, J=7.0 Hz, 1H), 2.25 (s, 3H), 1.60 (q, J=3.9 Hz, 2H), 1.41 (d, J=7.0 Hz, 3H), 1.21 (q, J=3.9 Hz, 2H). ESI-MS m/z calculated C₂₈H₂₇N₅O₃ [M+H]⁺ 482.22, found 481.91.

### c) Cell Culture

All media and cell culture reagents were purchased from WISENT Bioproducts (Quebec, Canada). Chinese hamster ovary (CHO)-K1 cell line was a kind gift from Rithwik Ramachandran (University of Western Ontario), and was cultured in F-12K medium supplemented with 10% fetal bovine serum and 1% streptomycin/penicillin at 37° C., 5% CO₂. The MDA-MB-231 was a kind gift from Simon Lees (NOSM University), and was cultured in Dulbecco's Modified Eagle Medium (DMEM) supplemented with 10% fetal bovine serum and 1% streptomycin/penicillin at 37° C., 5% CO₂.

### d) Overexpression of LPA₁ in CHO-K1 Cell Line

The plasmid encoding LPA₁-GFP was purchased from OriGene Technologies (Rockville, MD, USA). CHO-K1 cells were seeded onto 96-well plates at a density of 8000 cells/well and then transiently transfected with the plasmid using X-tremeGENE 9 DNA Transfection Reagent (Roche) in antibiotic-free media. Transiently transfected cells were assayed or imaged at 48 h post-transfection.

### e) CAMP Assay

CAMP levels were measured using CAMP-Glo™ Assay kit (Promega) according to the manufacture's instructions. CHO-K1 cells overexpressing LPA₁ were incubated with various concentrations of ⅕ log diluted test compounds, 100 nM forskolin, and 1 UM LPA in induction buffer (1×phosphate-buffered saline with 500 μM isobutyl-1-methylxanthine and 100 UM 4-(3-butoxy-4-methoxybenzyl) imidazolidone) for 30 min at room temperature. Changes in cAMP were measured using BioTek Synergy HTX plate reader.

### f) Colony Formation Assay

MDA-MB-231 cells were trypsinized and plated in 6-well plate at a density of 1,000 cells/well and then cultured in 10% FBS DMEM. Cells were allowed to attach overnight and then exposed to LPA or LPA with 1f (0, 0.5, 1.0 or 1.5 mM). Forty-eight hours after chemical treatment, the media was replaced with fresh media, and the plates were incubated at 37° C. Ten days later, the cells were fixed and stained with 4% paraformaldehyde in 0.1% crystal violet. The number of colonies, defined as >50 cells/colony were counted, and the number of colonies were calculated using ImageJ.

### g) Transwell Cell Migration and Invasion Assays

Transwell assays were conducted in 24-well transwell plates (pore size: 8 μm) to assess the migratory and invasive capacities of MDA-MB-231 cells. For migration assays, 2×10⁴ cells were placed in 200 μl of serum-free DMEM in the upper chamber and then 500 μl of DMEM containing 10% FBS was added to the lower chamber. For the invasion assays, the chamber inserts were precoated with 50 μl of 1:9 mixture of basement membrane extract (R&D Systems) and DMEM overnight in a 37° C. incubator, then 6×10⁴ cells were seeded in the upper chamber. 500 μl of DMEM containing 10% FBS was added to the lower chamber. Cells were treated with LPA or LPA with 1f (0, 0.5, 1.0 or 1.5 mM) for 48 h. 4% paraformaldehyde was used to fix the cells that had migrated or invaded to the lower surface of the membrane. Crystal violet (0.1%) was applied for staining the fixed cells for 15 min. Five random 100×microscopic fields were selected to count the stained cells.

### h) Wound Healing Assays

Cells were seeded in a six-well plate at a density of 3×10⁵ cells/well. When the cells formed a tight cell monolayer, a 200-μl plastic pipette tip was used to make a scratch. Cells were washed with PBS to remove cell debris, cultured in serum-free DMEM medium and exposed to LPA or LPA with 1f (0, 0.5, 1.0 or 1.5 mM) for 48 h. Wound photographs were recorded at the indicated times and analyzed by ImageJ software. All assays were conducted three times in our study.

The scope of the claims should not be limited by the preferred embodiments set forth in the examples but should be given the broadest interpretation consistent with the description as a whole.

TABLE 1
the 1^{st} series of triazole carbamate derivatives with IC50 values.

| Compounds | IC₅₀ (nM) | % inhibition |
|---|---|---|
| RO-2 | 18.3 | 111.6 |
| 1b | 3337 | 58.43 |
| 1c | 2145.5 | 77.1 |
| 1d | 1487.6 | 81.9 |
| 1e | 93.3 | 102.1 |
| 1f | 16.0 | 109.8 |
| 1g | 192.7 | 96.4 |
| 1h | 91.2 | 115.7 |
| 1i | NE | |

TABLE 2
the 2nd series of triazole urea derivatives with IC50 values.

| Compounds | IC₅₀ (nM) | % inhibition |
|---|---|---|
| 2a | 1004.6 | 41.8 |
| 2b | 3152 | 40.5 |
| 2c | 1998 | 88.1 |
| 2d | 4468.5 | 49.5 |
| 2e | 780.2 | 102.0 |
| 2f | 242.4 | 108.1 |
| 2g | 299.2 | 92.7 |
| 2h | 239.7 | 83.8 |

1. A compound comprising a chemical structure according to Formula (I) or (II): 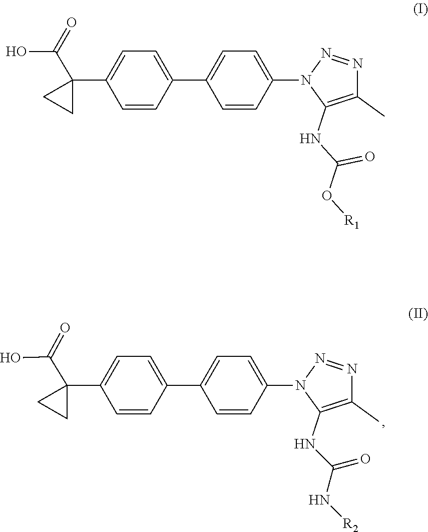
wherein R₁ and R₂ are independently a fluorocumene or a fluoroethylbenzene.

2. The compound according to claim 1, wherein R₁ or R₂ is independently selected from the group consisting of:

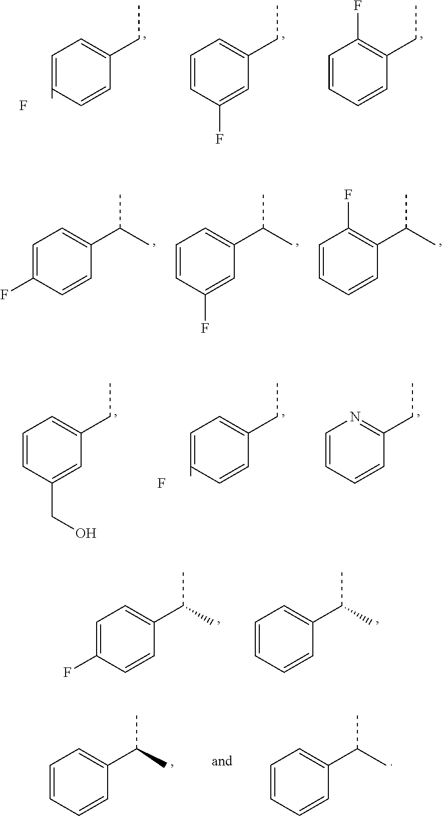

3. The compound according to claim 1, wherein the compound comprises a chemical structure as set forth in Formula (I) and R₁ is selected from the group consisting of:

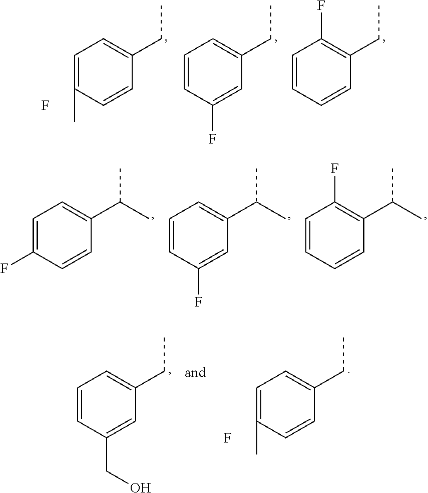

4. The compound according to claim 1, wherein the compound comprises a chemical structure as set forth in Formula (II) and R₂ is selected from the group consisting of:

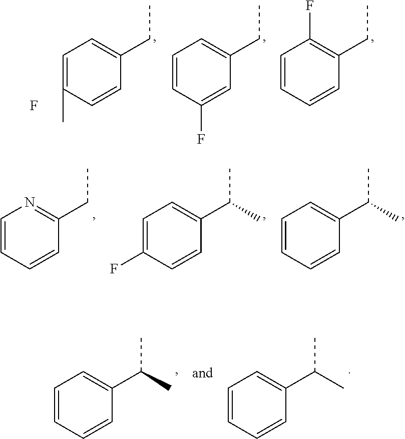

5. The compound according to claim 1 wherein the compound comprises a chemical structure as set forth in Formula (I) and R₁ is

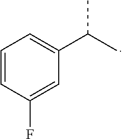

6. The compound according to claim 1, wherein, R₁ or R₂ is independently selected from the group consisting of:

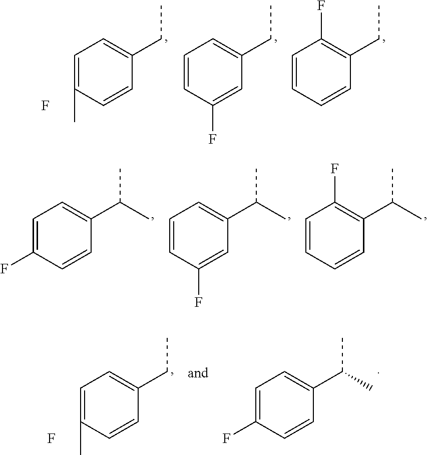

7. The compound according to claim 6 wherein the F is Fluorine-18.

8. A method of treating an LPA₁-related pathological condition comprising administering an effective amount of a compound according to claim 1 to an individual in need of such treatment.

9. The method according to claim 8 wherein the LPA₁-related pathological condition is selected from the group consisting of breast cancer, glioblastoma, cervical cancer, and Idiopathic Pulmonary Fibrosis.
